(19) United States
(12) Patent Application Publication
(10) Pub. No.: US 2008/0292481 A1
(43) Pub. Date: Nov. 27, 2008

(54) MASTIC PUMP

(76) Inventors: David J. Castagnetta, JR., Lawrenceville, GA (US); Collin Sobczynski Farill, Decatur, GA (US); Erick Eladio Rios, Atlanta, GA (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 11/753,293

(22) Filed: May 24, 2007

(51) Int. Cl. A61M 1/00; F04B 53/12

(52) U.S. Cl. 417/437; 417/554

(57) ABSTRACT

A mastic pump is provided. The mastic pump is operable to pump mastic from a container to a mastic application tool. The mastic pump includes a pump body, a piston movable within the pump body, an actuator coupled to the piston for moving the piston, and a fulcrum adjustor coupled to the actuator at one of at least two positions. The mastic pump may include a valve diaphragm having an opening defined therein to allow mastic flow. Also, the pump may include a foot stand coupled to the pump body having a concave shaped portion. In addition, the actuator may include a grip and a carrying member spaced from the grip, the carrying member is grippable to transport the pump. Further, the mastic pump may include an accessory connector coupled to the pump body for connecting and disconnecting a mastic tool accessory to the pump body without the use of tools.

### FIELD OF THE INVENTION

The present invention relates to a mastic pump and, more particularly, to a mastic pump for filling a variety of drywall taping tools with mastic pumped from a mastic filled container.

### BACKGROUND

Mastic pumps are used to pump mastic from a mastic filled container to a mastic application tool connected to the pump. A variety of mastic application tools can connect to the pump for filling. To enable connection of a variety of mastic application tools to the pump, a variety of accessories are removably connectable to the pump and each mastic application tool is connectable to its respective accessory. Such accessories are connectable to the mastic pumps by fasteners requiring tools such as a wrench, screwdriver, pliers, etc. to tighten and loosen the fasteners.

Mastic pumps typically include a pump body positionable in the mastic filled container, a valve positioned at the bottom of the pump body and submerged in mastic when the pump body is positioned in the container, a piston positioned inside the pump body, a handle connected to the piston and actuatable by a user to reciprocate the piston, and a foot stand positioned externally of the mastic filled container and operable to be stepped upon by a user. Such mastic pumps operate in a single condition and pump mastic therefrom at a single speed or volume.

Typical mastic pumps also include a diaphragm in the valve. Such diaphragms are solid, have a circular edge or periphery, and provide very little space between the circular periphery of the diaphragm and the interior surface of the valve. Through such limited space, mastic passes the diaphragm and into the pump. This small amount of space provides low, undesirable mastic flow rates during pumping. Such valves may also be removable from the pump body for cleaning or replacement purposes. In order to remove such valves, a user must use a tool such as, for example, a wrench, screw driver, pliers, etc. Accordingly, a user is required to carry or locate such tools and perform the time consuming task of removing the valve with the tools.

These types of mastic pumps also provide undesirable effects when transported. Such mastic pumps are usually carried by gripping an outward end of the handle and lifting the mastic pump by the end of the handle. Lifting and transporting the mastic pump in this manner is awkward and places a lot of stress on a user's hand, wrist, and arm.

Also, foot stands of these typical mastic pumps are provided for a user to step thereupon and are meant to establish stability of the mastic pump during operation of the mastic pump. In order to provide a rigid and sturdy foot stand, the foot stand must be made of a relatively large quantity of material that will provide the necessary rigidity and sturdiness, thereby providing a heavy foot stand. Other typical foot stands include ribbing to provide sufficient rigidity and sturdiness. The extra material provided by this ribbing provides a heavy foot stand. In addition, such foot stands provide a small stepping portion on which a user may step. This small stepping area does not provide the necessary stability for the mastic pump both during operation and while standing unattended.

Accordingly, a need exists for a mastic pump that satisfies at least these and other deficiencies of conventional mastic pumps.

### SUMMARY

In some aspects, a mastic pump is provided and includes a pump body for holding mastic, a piston at least partially positioned in the pump body and movable within the pump body, an actuator coupled to the piston for moving the piston, and a fulcrum adjustor coupled to the actuator at one of at least two positions.

In some aspects, a mastic pump is provided and includes a pump body for holding mastic, a piston at least partially positioned within the pump body and movable within the pump body, an actuator coupled to the piston for moving the piston, and a valve diaphragm coupled to the pump body and having an opening defined therein to allow mastic flow.

In some aspects, a mastic pump is provided and includes a pump body at least partially positionable in a mastic filled container and a foot stand coupled to the pump body and including a stepping portion and an upright portion extending vertically upward from the stepping portion, the upright portion including an interior surface that is at least partially concave.

In some aspects, a mastic pump is provided and includes a pump body for holding mastic, a piston at least partially positioned in the pump body and movable within the pump body, and an actuator coupled to the piston and movable between a first position and a second position to move the piston, the actuator including a grip that is grippable by a user to move the actuator between the first and second positions, the actuator also including a carrying member spaced from the grip that is grippable by a user to transport the pump.

In some aspects, a mastic pump is provided and includes a pump body for holding mastic, a piston at least partially positioned in the pump body and movable within the pump body, an actuator coupled to the piston for moving the piston, and an accessory connector coupled to the pump body for connecting and disconnecting a mastic tool accessory to the pump body without the use of tools.

Other aspects of the invention will become apparent from consideration of the detailed description, accompanying drawings, and claims.

### BRIEF DESCRIPTION OF THE DRAWINGS

FIG. 1 is a perspective view of a mastic pump shown with a mastic tool accessory connected to the pump.

FIG. 2 is another perspective view of the mastic pump illustrated in FIG. 1.

FIG. 3 is a perspective view of the mastic pump illustrated in FIG. 1, shown with a container for holding mastic.

FIG. 4 is another perspective view of the mastic pump illustrated in FIG. 1, shown with a handle of the pump in a downward position.

FIG. 5 is an elevational view of a portion of the mastic pump illustrated in FIG. 1 with a foot valve of the mastic pump shown in a locked condition.

FIG. 6 is an elevational view similar to FIG. 5 with the foot valve shown in an unlocked condition.

FIG. 7 is a bottom view of the foot valve and an adapter of the mastic pump illustrated in FIG. 1.

FIG. 8 is a top view of the foot valve and adapter of the mastic pump illustrated in FIG. 1.

FIG. 9 is an exploded view of the foot valve of the mastic pump illustrated in FIG. 1.

FIG. 10 is a perspective view of a piston of the mastic pump illustrated in FIG. 1 with the piston shown in a closed condition.

FIG. 11 is a perspective view of the piston of the mastic pump illustrated in FIG. 1 with the piston shown in an open position.

FIG. 12 is a perspective view of an accessory connector of the mastic pump illustrated in FIG. 1 with the accessory connector shown in a locked condition.

FIG. 13 is a partially exploded view of the accessory connector of the mastic pump illustrated in FIG. 1 with the accessory connector shown in an unlocked condition and an accessory removed from the mastic pump.

FIG. 14 is a perspective view of a foot stand of the mastic pump illustrated in FIG. 1.

FIG. 15 is a bottom view of the foot stand illustrated in FIG. 14.

FIG. 16 is a perspective view of a fulcrum adjustor and a fulcrum of the mastic pump illustrated in FIG. 1.

FIG. 17 is an exploded view of the fulcrum adjustor shown in FIG. 16.

FIG. 18 is a cross-sectional view taken along line 18-18 in FIG. 16 through the fulcrum adjustor, the fulcrum adjustor is shown in a fast condition.

FIG. 19 is a cross-sectional view similar to FIG. 18, the fulcrum adjustor is shown in an unlocked condition and in the position occupied by the fulcrum adjustor when in the fast condition.

FIG. 20 is a cross-sectional view similar to FIG. 18, the fulcrum adjustor is shown in an unlocked condition and in the position occupied by the fulcrum adjustor when in an easy condition.

FIG. 21 is a cross-sectional view similar to FIG. 18, the fulcrum adjustor is shown in the easy condition.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

### DETAILED DESCRIPTION

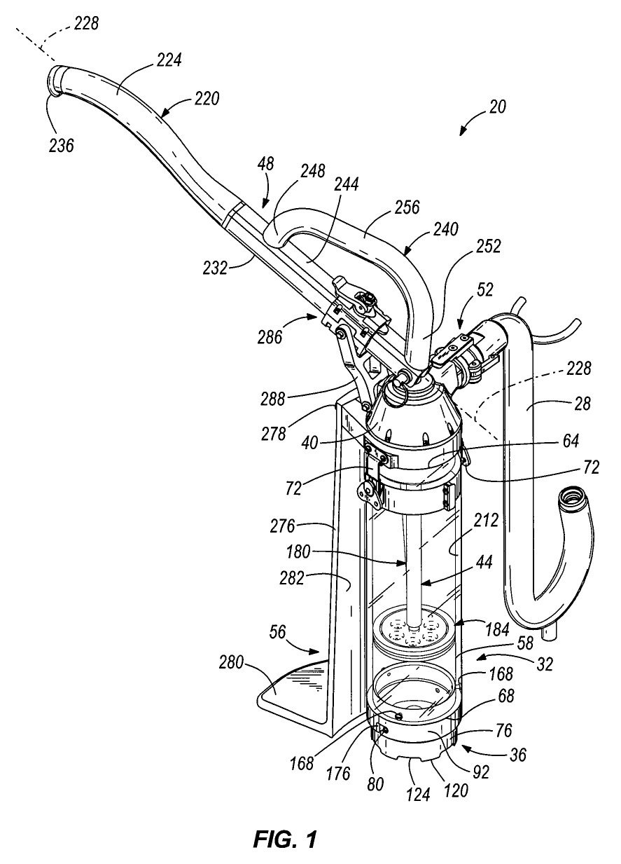

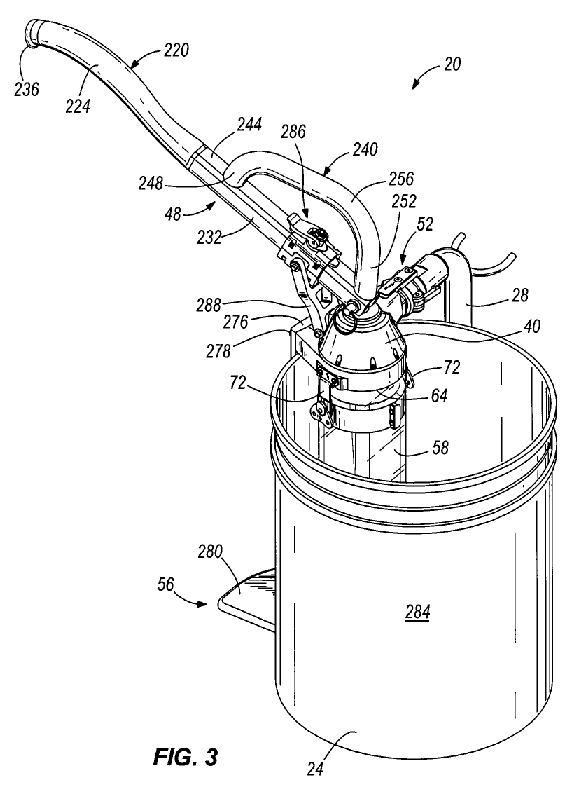

Referring to FIGS. 1-4, a mastic pump 20 is illustrated and is manipulatable by a user to pump mastic, otherwise known as mud, from a mastic filled container 24 (see FIG. 3) to any one of a variety of mastic application tools removably couplable to the mastic pump 20. A variety of accessories 28 are removably couplable to the mastic pump 20 to connect a variety of mastic application tools to the mastic pump 20. In the illustrated embodiment and for exemplary purposes, an accessory 28 commonly referred to as a “goose neck” is coupled to the mastic pump 20 and enables a particular type of mastic application tool, such as that shown in U.S. Pat. No. 6,874,557, to couple to the mastic pump 20.

With continued reference to FIGS. 1-4, the mastic pump 20 includes a pump body 32, a foot valve 36, a piston 44, an actuator or handle 48, an accessory connector 52, a foot stand 56, a fulcrum adjustor 286, and a fulcrum 288. The pump body 32 includes a cylindrical tube 58, a pump head 40, and an adapter 92. The tube 58 can be made of a large variety of materials including PVC, aluminum, stainless steel, or any other type of rigid plastic or environmentally-resistant metal. The tube 58 can also be made of material that is clear, transparent, translucent, or opaque. The pump head 40 is connected to a top end 64 of the tube 58 via latches 72, and the adapter 92 is connected to a bottom end 68 of the tube 58 via fasteners 168. The foot valve 36 is removably connected to the adapter 92 of the pump body 32. In some embodiments, the tube 58 can have a diameter ranging from about 2.25 inches to about 3.75 inches. Preferably, the tube 58 has a diameter of about 3.5 inches. Also, the pump 20 has a sufficient height to accommodate various sized mastic filled containers 24 such as, for example, a 5-gallon bucket and a painter's bucket.

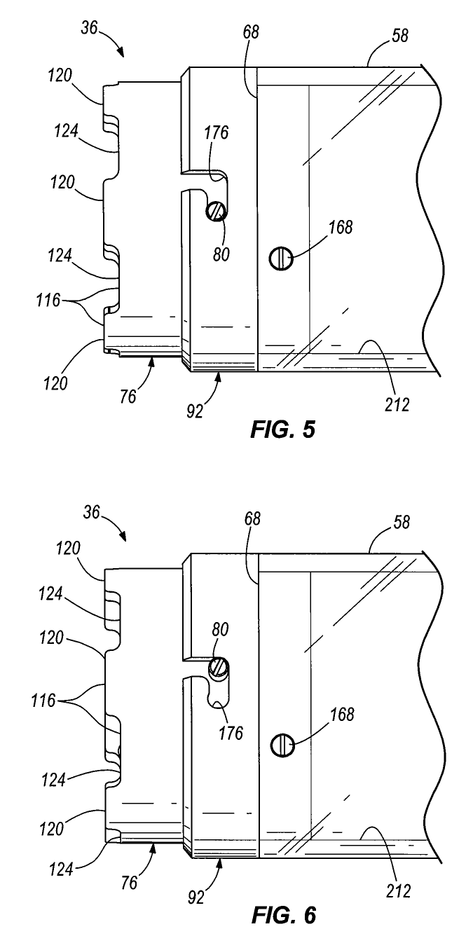

With reference to FIG. 5, the foot valve 36 is shown in a locked condition, in which the foot valve 36 is connected to the adapter 92 of the pump body 32 and cannot be inadvertently removed. With reference to FIG. 6, the foot valve 36 is shown in an unlocked condition, in which the foot valve 36 can be removed from the adapter 92. In the illustrated embodiment, the foot valve 36 is moved between the locked and unlocked conditions by manually twisting the foot valve 36. Accordingly, the foot valve 36 is connectable to and removable from the pump body 32 of the mastic pump 20 without the use of tools. Alternatively, the foot valve 36 may be moved between the locked and unlocked conditions in other manners without the use of tools.

Referring now to FIGS. 5-9, the foot valve 36 includes a valve body 76, a pair of locking pins 80, a screen 84, a screen retainer 88, a valve diaphragm 100, a guide post 104, and a pair of fasteners 108 for connecting the guide post 104 to the valve body 76. The valve body 76 defines a pair of apertures 112 therein for receiving the locking pins 80. The locking pins 80 can be connected to the valve body 76 in a variety of manners such as, for example, press-fit, threading, detent and groove, etc. Alternatively, the locking pins 80 can be unitarily formed with the valve body 76. In the illustrated embodiment, the locking pins 80 are disposed on opposite sides of the valve body 76 approximately 180° from one another. Alternatively, the pair of locking pins 80 may be disposed in a different orientation on the valve body 76 relative to one another. Also in the alternative, the foot valve 36 may include more than two locking pins 80 for connecting the foot valve 36 to the pump body 32 of the mastic pump 20. The valve body 76 is cylindrical in shape, has a corrugated bottom edge 116 comprised of alternating projections 120 and cavities 124, and has three openings 128 defined in a bottom surface 132 of the valve body 76. The bottom surface 132 has three spokes 136 separating the three openings 128. The alternating projections 120 and cavities 124 defined in the bottom edge 116 of the valve body 76 allow mastic to pass the exterior wall 140 of the valve body 76 when the bottom edge 116 of the valve body 76 engages a bottom surface of the mastic container 24.

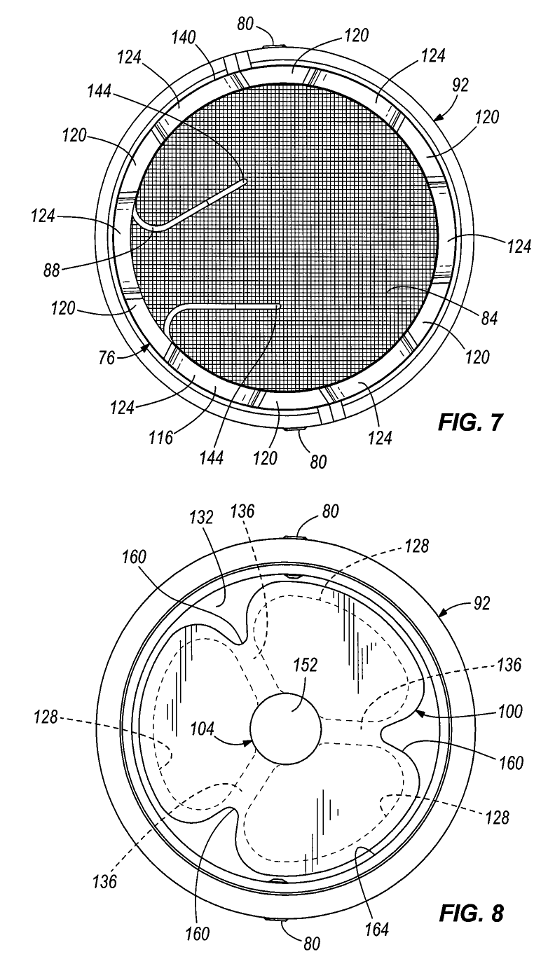

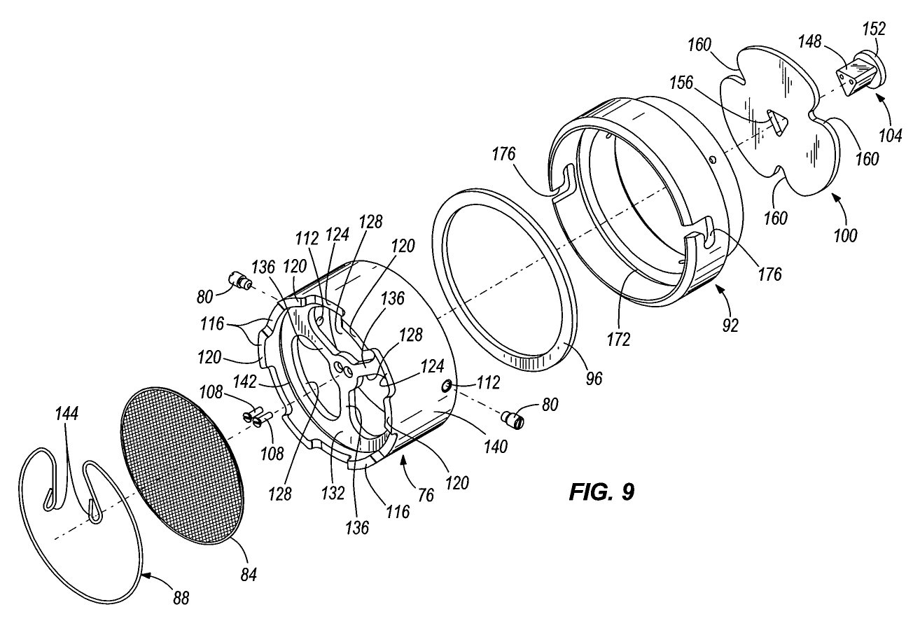

With particular reference to FIGS. 7 and 9, the screen 84 is removably connected to the valve body 76 with the screen retainer 88. The screen 84 filters the mastic pumped by the mastic pump 20 by inhibiting large clumps of mastic (usually dried or semi-dried pieces of mastic) or other debris present in the mastic from entering the pump 20. The screen 84 can have varying mesh sizes in order to filter varying sizes of debris. The screen 84 is removable to facilitate easy cleaning of the screen 84 and the foot valve 36, and to facilitate replacement of a damaged or inoperative screen. The screen retainer 88 is a wire ring that is positionable in a groove 142 along with the screen 84 to resiliently retain the screen 84 in the groove 142 and connect the screen 84 to the valve body 76. To remove the screen retainer 88, a user simply grasps ends 144 of the screen retainer 88, pinches the ends 144 together, and pulls the screen retainer 88 away from the screen 84 and out of the groove 142. The screen 84 can now be removed from the groove 142 and the valve body 76.

Referring to FIG. 9, the guide post 104 is connected to the valve body 76 via fasteners 108 and includes a shaft 148 and a stop surface 152. The diaphragm 100 defines an aperture 156 therein and the post 104 is positioned in the aperture 156. During operation of the foot valve 36, the diaphragm 100 slides vertically along the shaft 148 with upward movement of the diaphragm 100 limited by the stop surface 152, and downward movement of the diaphragm 100 limited by the bottom surface 132 of the valve body 76. In the illustrated embodiment, the shaft 148 is triangular shaped and the aperture 156 is complementarily shaped to facilitate vertical movement of the diaphragm 100 relative to the guide post 104 and to inhibit rotational movement of the diaphragm 100 relative to the guide post 104. Alternatively, the shaft 148 and the aperture 156 can assume different complementary shapes as long as vertical movement is facilitated and rotational movement is inhibited.

With particular reference to FIG. 8, the diaphragm 100 includes openings 160 defined in an edge or periphery thereof. In the illustrated embodiment, the diaphragm 100 includes three openings 160, but the diaphragm 100 may include any number of openings 160 defined in the edge thereof and still be within the spirit and scope of the present invention. Also, the openings 160 may be defined in the diaphragm 100 in locations different than the edge such as, for example, internally of the edge, and can have a variety of shapes such as, for example, circular, triangular, or any other polygonal shape. The openings 160 facilitate sufficient space so that mastic can flow easily past the diaphragm 100. In the illustrated embodiment, the openings 160 provide sufficient space between the edge of the diaphragm 100 and an interior surface 164 of the valve body 76. In some embodiments, the diaphragm 100 is made of polyurethane. In other embodiments, the diaphragm 100 is made of other types of plastics or environmentally-resistant metals.

With continued reference to FIGS. 5-9, the adapter 92 is connected to the bottom end 68 of the tube 58 via fasteners 168 (also see FIGS. 1, 2, and 4). A gasket 96 is disposed in the adapter 92 and is supported by a lip 172 defined in the adapter 92. When the valve body 76 is connected to the adapter 92, the gasket 96 is compressed between the valve body 76 and the adapter 92 to create a seal between the valve body 76 and the adapter 92. The adapter 92 includes a pair of L-shaped grooves 176 therein for removably receiving the locking pins 80 connected to the valve body 76. Alternatively, the grooves 176 can assume a shape different than the illustrated L-shape as long as the groove and locking pin connection is sufficient to connect the foot valve 36 to the pump body 32 of the pump 20. Furthermore, the adapter 92 can include more than two grooves 176 for receiving a complementary number of locking pins 80. Ultimately, it is desirable to have matching numbers of locking pins 80 and grooves 176.

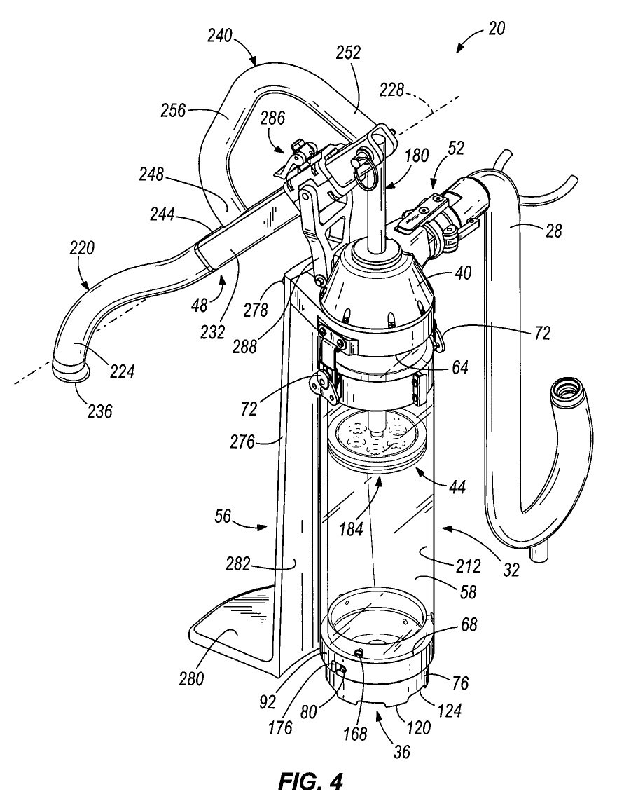

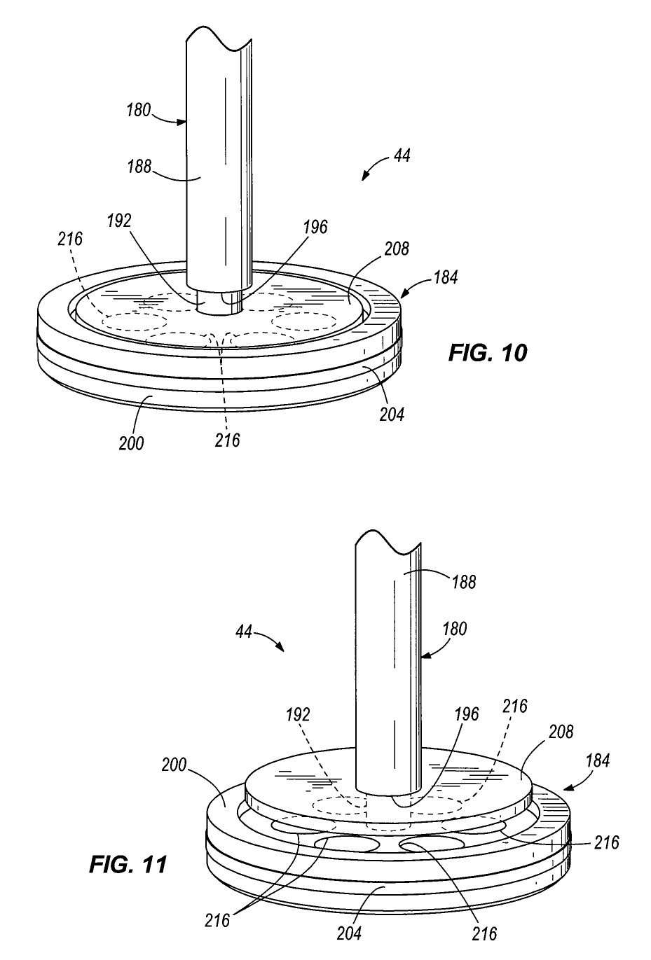

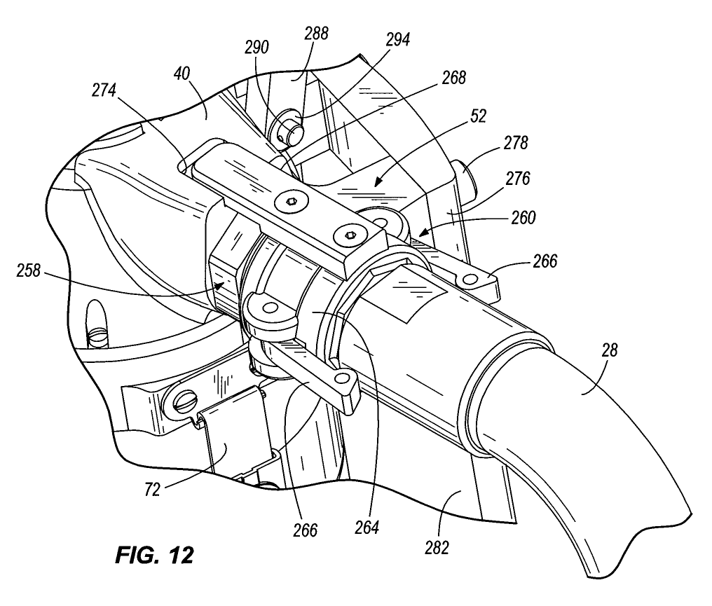

Referring back to FIGS. 1 and 4, the handle 48 is movable in an upward and downward pumping motion to reciprocate the piston 44 between a fully upward position and a fully downward position. FIG. 1 illustrates the handle 48 in a fully upward position and the piston 44 in an associated fully downward position, and FIG. 4 illustrates the handle 48 in a fully downward position with the piston 44 in an associated fully upward position. With additional reference to FIGS. 10 and 11, the piston 44 includes a piston shaft 180 and a piston valve 184 connected to the piston shaft 180. The piston shaft 180 includes a first portion 188 having a first diameter and a second portion 192 having a second diameter smaller than the first diameter. At the transition from the first portion 188 to the second portion 192, a stop surface 196 is created. The piston valve 184 includes a piston body 200, a seal 204 supported by the piston body 200, and a piston diaphragm 208. In some embodiments, the seal 204 is made of Teflon-impregnated fiberglass, the piston diaphragm 208 is made of polyurethane, and the piston body 200 is made of aluminum. In other embodiments, the seal 204, the piston diaphragm 208, and the piston body 200 are made of different materials. The seal 204 engages an interior surface 212 of the tube 58 and inhibits mastic from passing the piston 44 between the valve body 76 and the interior surface 212. A plurality of apertures 216 are defined in the piston body 200 to allow mastic to pass through the apertures 216 and by the piston valve 184. The piston diaphragm 208 is slidably supported on the second portion 192 of the piston shaft 180 and moves between a closed position (see FIG. 10), in which the piston diaphragm 208 is fully down and engages the piston body 200 to cover or block the apertures 216, and an open position, in which the diaphragm 208 is fully upward and engages the stop surface 196. In the closed position, the piston diaphragm 208 inhibits mastic from passing through the apertures 216. In the open position, the piston diaphragm 208 allows mastic to pass through the apertures 216 and by the piston valve 184. Operation of the piston valve 184 in association with the foot valve 36 will be described in greater detail below.

Referring to FIGS. 1-4, the handle 48 includes an ergonomically shaped grip 220 that a user grasps to actuate the handle 48. The grip 220 has an arcuate portion 224 providing a user with a comfortable hold on the grip 220. A portion of the handle 48 is straight and a longitudinal axis 228 of the handle 48 is defined through a center of the straight portion 232. The grip 220 at least partially curves away from the longitudinal axis 228 to form the arcuate portion 224. The handle 48 also includes a stop member 236 at an outward end thereof to inhibit a user's hand from sliding off the end of the handle 48 and to provide support to the user's hand during actuation of the handle 48. The handle 48 further includes a carrying member 240 extending upward from a top surface 244 of the handle 48. A user can grasp the carrying member 240 to transport the mastic pump 20 from one location to another location. The carrying member 240 includes a short leg 248, a long leg 252, and an intermediate member 256 extending between the short and long legs 248, 252 that can be gripped by a user. The intermediate member 256 is non-parallel and acutely angled relative to the handle axis 228. The carrying member 240 is configured such that when a user grips the intermediate member 256 and lifts the mastic pump 20, the handle 48 is capable of only moving in an upward direction, which only allows the piston 44 to move in a downward direction. Since the piston 44 does not move vertically upward during transport of the pump 20, mastic is not undesirably emitted from the mastic pump 20. Also, the carrying member 240 is at least partially positioned over the body 32 and foot stand 56 of the pump 20. The body 32 and foot stand 56 provide the majority of the weight of the pump 20 and by positioning the carrying member 240 over these components, the majority of the weight is disposed directly underneath the carrying member 240. Accordingly, carrying the pump 20 by the carrying member 240 is very ergonomic.

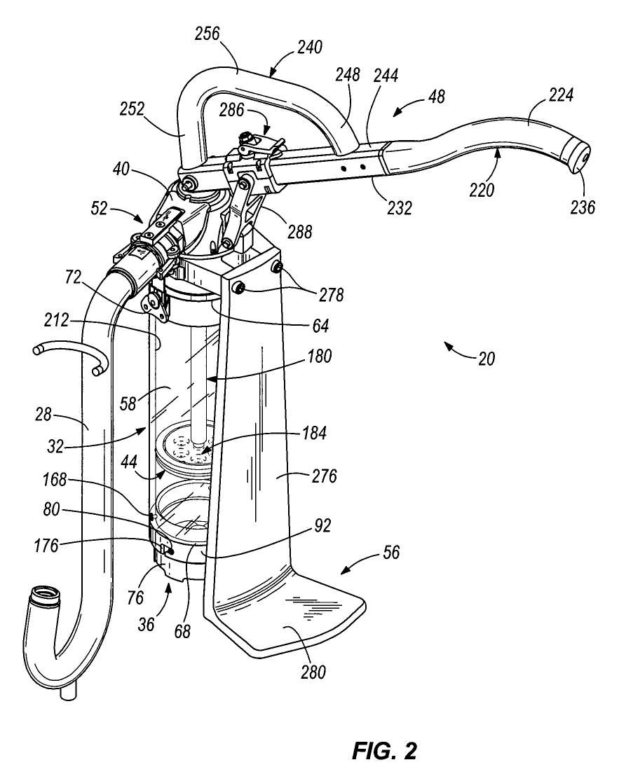

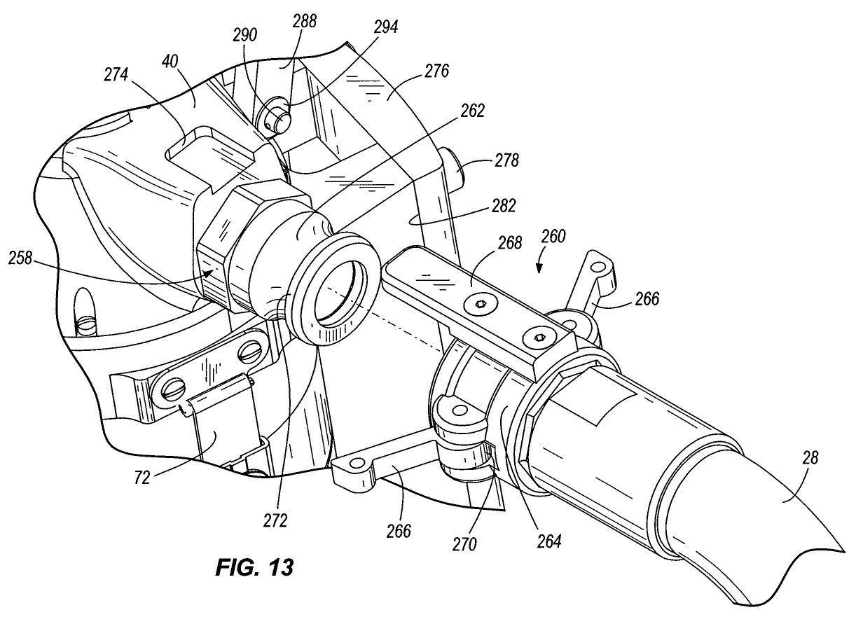

Referring now to FIGS. 2, 12, and 13, the accessory connector 52 removably connects an accessory 28 to the mastic pump 20. The accessory 28 enables connection of a mastic application tool to the pump 20 without the use of tools. A wide variety of accessories 28 can include an accessory connector 52, thereby enabling a large variety of accessories 28 to connect to the mastic pump 20 and, therefore, enabling a large variety of mastic application tools to connect to the mastic pump 20. For exemplary purposes, only one type of accessory 28 is illustrated. The accessory connector 52 includes a nozzle 258 connected to the pump 20 and an accessory portion 260 connected to the accessory 28. The nozzle 258 is threadably connected to the pump head 40 and remains connected thereto whether the accessory 28 is connected to or disconnected from the pump 20. The accessory portion 260 removably connects to the nozzle 258 to removably connect the accessory 28 to the pump 20. The nozzle 258 includes a groove 262 extending around it, and the accessory portion 260 includes a connector housing 264 shaped for receiving the nozzle 258 therein, a pair of cam levers 266 pivotally connected to the connector housing 264, and a locking projection or tab 268 connected to the housing 264. With particular reference to FIG. 13, the accessory portion 260 is disconnected from the nozzle 258. To connect the accessory portion 260 to the mastic pump 20, the connector housing 264 is aligned with the nozzle 258, the housing 264 is positioned over the nozzle 258, and the cam levers 266 are pivoted to the position shown in FIG. 12. When the cam levers 266 are in the position illustrated in FIG. 12, a portion 270 of the cam levers 266 engage the nozzle 258 in the groove 262 and a lip 272 of the nozzle 258 prevents the accessory portion 260 from being pulled off of the nozzle 258. To remove the accessory portion 260 from the nozzle 258, the cam levers 266 are pivoted to the position shown in FIG. 13 and the accessory 28 is pulled off of the nozzle 258. To inhibit rotational movement of the accessory 28 relative to the mastic pump 20 during operation of the mastic pump 20, the locking tab 268 is positioned in a tab receptacle 274 defined in the pump head 40. Rotational movement of the accessory 28 relative to the mastic pump 20 is limited in both directions by engagement of the locking tab 268 with edges of the tab receptacle 274. The tab 268 and receptacle 274 can assume a wide variety of shapes as long as they interact to inhibit rotational movement of the accessory 28 relative to the pump 20.

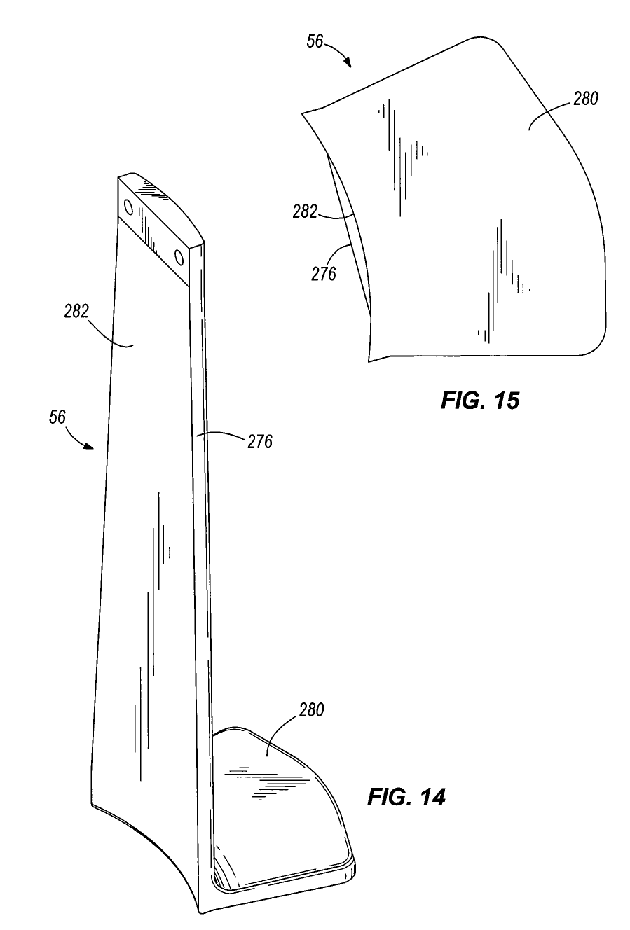

Referring now to FIGS. 1-4, 14, and 15, the foot stand 56 is illustrated and provides a surface on which a user may step during operation of the mastic pump 20 to provide stability. The foot stand 56 includes an upright portion 276 connected to the pump head 40 via fasteners 278 and a horizontal stepping portion 280 on which a user steps. The upright portion 276 has a concave shape, which increases rigidity of the foot stand 56 without the use of ribbing or additional material. The stepping portion 280 has a large footprint, which provides good stability for the mastic pump 20 during operation and while the mastic pump 20 is standing unattended.

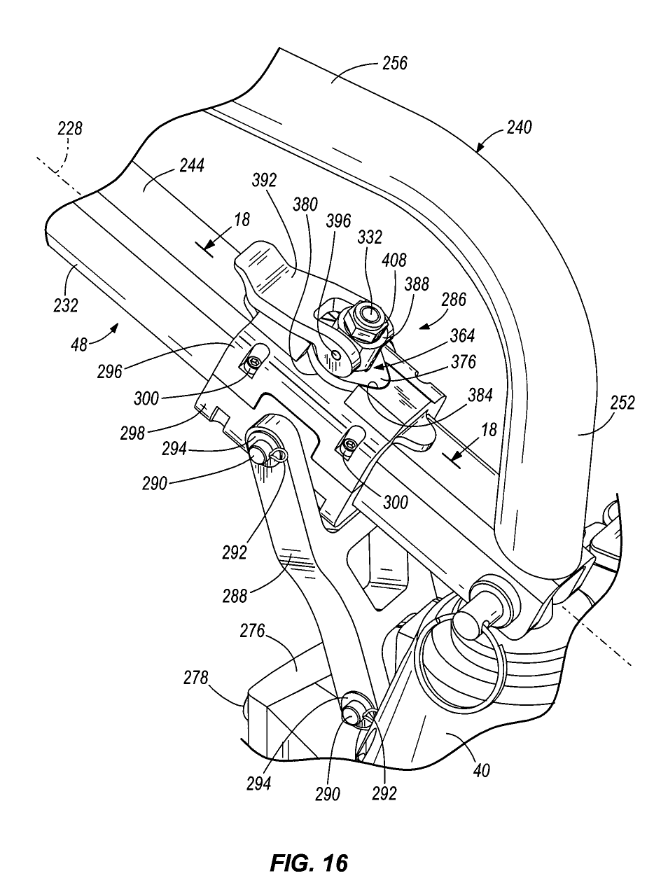

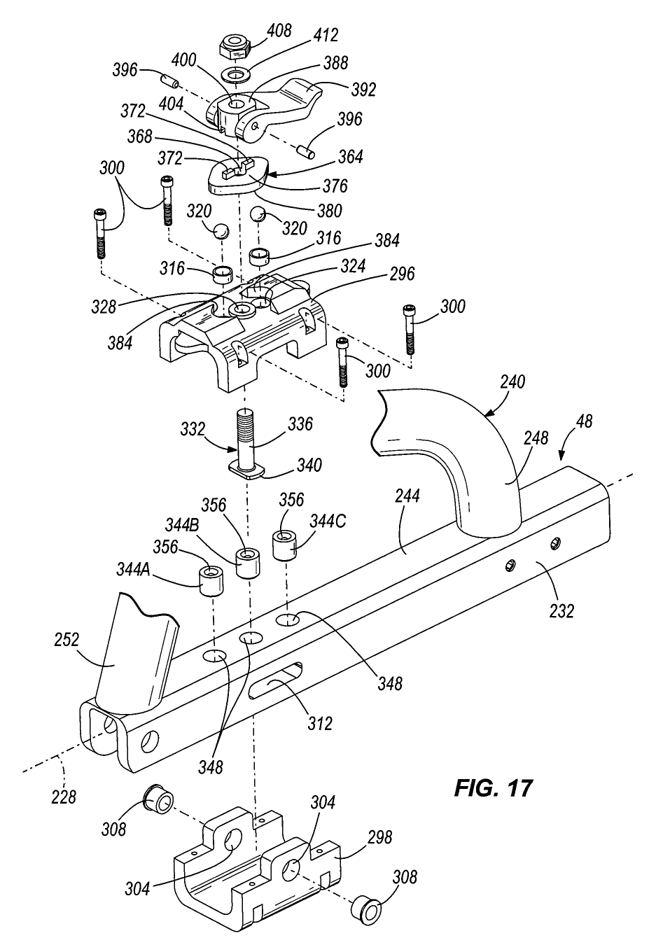

With reference to FIGS. 1-4 and 16-21, the fulcrum adjustor 286 is illustrated and enables a user to adjust the fulcrum 288 relative to the handle 48, thereby adjusting the leverage of the handle 48. The fulcrum 288 is pivotally connected between the fulcrum adjustor 286 and the pump head 40. The fulcrum 288 is connected to the fulcrum adjustor 286 and the pump head 40 with a shaft 290, cotter pins 292, and washers 294. Other manners of connecting the fulcrum 288 to the fulcrum adjustor 286 and the pump head 40 are possible and are within the spirit and scope of the present invention. The fulcrum adjustor 286 includes an upper housing 296 and a lower housing 298 connected to each other around the handle 48 via fasteners 300. In some embodiments, the upper and lower housings 296, 298 are made of aluminum. In other embodiments, the upper and lower housings 296, 298 are made of different metals or plastic. The lower housing 298 defines a pair of apertures 304 therein for receiving bushings 308. The shaft 290 used to connect the upper end of the fulcrum 288 to the fulcrum adjustor 286 is supported by the bushings 308 and extends through a slot 312 defined in the handle 48. The slot 312 allows the shaft 290 to move relative to the handle 48 without interference (discussed in greater detail below). The fulcrum adjustor 286 also includes a pair of bushings 316 and a pair of balls 320 supported in the bushings 316. The bushings 316 and balls 320 are positioned in a pair of apertures 324 defined in the upper housing 296. The upper housing 296 also defines a post aperture 328 through which a post 332 of the fulcrum adjustor 286 extends. The post 332 includes a cylindrical member 336 and a head 340. The head 340 is trapped between the upper housing 296 and a top surface 244 of the handle 48 to secure the post 332 in place. The cylindrical member 336 of the post 332 extends upward through the post aperture 328 defined in the upper housing 296. The fulcrum adjustor 286 further includes three cups 344A, 344B, 344C positioned in three holes 348 defined in the handle 48. Each cup has a cavity defined there through which includes a larger diameter frusto-conical portion or ball receiving opening 356 near a top of the cup and a smaller diameter portion 360 extending downward from the frusto-conical portion 356 to the bottom of the cup. The larger diameter frusto-conical portions 356 of the cups 344 are capable of receiving the balls 320 therein.

With further reference to FIGS. 1-4 and 16-21, the fulcrum adjustor 286 further includes a plate 364 defining an opening 368 in which the post 332 is positioned. A pair of key projections 372 extend upward from an upper surface 376 of the plate 364 and a bottom surface 380 of the plate 364 engages the balls 320. The plate 364 is positioned in a plate receptacle 384 defined in the upper housing 296 and the plate receptacle 384 is shaped complementary to the plate 364 to inhibit rotational movement of the plate 364 relative to the upper housing 296 during manipulation of the fulcrum adjustor 286. The fulcrum adjustor 286 also includes a lever post 388 and a lever 392. The lever 392 is connected to the lever post 388 with a pair of press pins 396 and is pivotally moveable about the pins 396 between an upward unlocked position and a downward locked position. The lever post 388 includes a post aperture 400 and a pair of key slots 404 in a bottom surface thereof. The post 332 is positioned in the post aperture 400 and the key projections 372 are positioned in the key slots 404. The engagement of the key projections 372 and the key slots 404 inhibits rotational movement between the lever post 388 and the plate 364 during manipulation of the fulcrum adjustor 286. A nut 408 is threaded onto an end of the post 332 and a washer 412 disposed between the nut 408 and a top surface of the lever post 388.

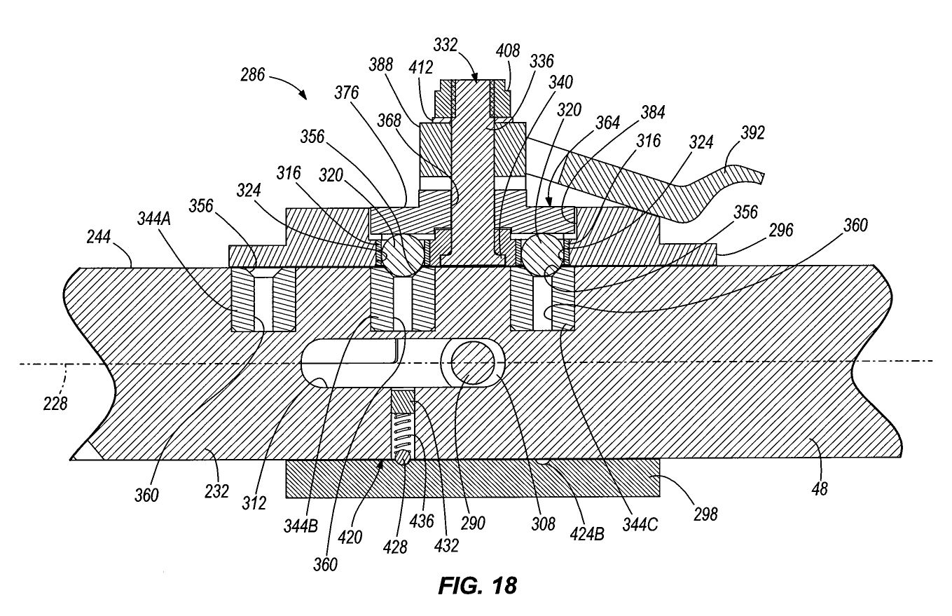

With reference to FIGS. 18-21, operation of the fulcrum adjustor 286 will be described. FIG. 18 illustrates the fulcrum adjustor 286 in a first or fast condition. The lever 392 is in its downward, locked position to lock the fulcrum adjustor 286 in the fast condition. By having the lever 392 positioned downward, the lever 392 applies a downward force on the plate 364, which applies a downward force on the balls 320. The downward force on the balls 320 forces the balls 320 into the ball receiving openings 356 of the second cup 344B and the third cup 344C. This downward force is sufficient to retain the fulcrum adjustor 286 in the fast condition. Equilibrium of the fulcrum adjustor 286 is achieved through opposing this downward force with an equal retaining or upward force traced along the following path: lever post 388, washer 412, nut 408, post 336, flange 340, and upper housing 296. With the balls 320 in the second and third cups 344B, 344C, the fulcrum 288 is in the furthest outward position on the handle 48, thereby providing the shortest leverage portion of the handle 48 possible and the maximum stroke length of the piston 44 possible. The shortest leverage portion requires the greatest amount of force to actuate the handle 48. However, the maximum piston stroke emits the most mastic per handle stroke. In the fast condition, fewer strokes of the handle 48 are required to fill a mastic application tool, thereby filling the mastic application tool faster. Appropriately, this condition is called the “fast” condition. A complete stroke of the handle 48 and piston 44 in the fast condition emits approximately 38 cubic inches of mastic. As an example, reference is made to the mastic application tool disclosed in U.S. Patent No. 6,874,557. Approximately five complete strokes of the handle 48 will fill such a mastic application tool when the fulcrum adjustor 286 is in the fast condition. Other mastic application tools may require more or less than five complete strokes in order to fill them.

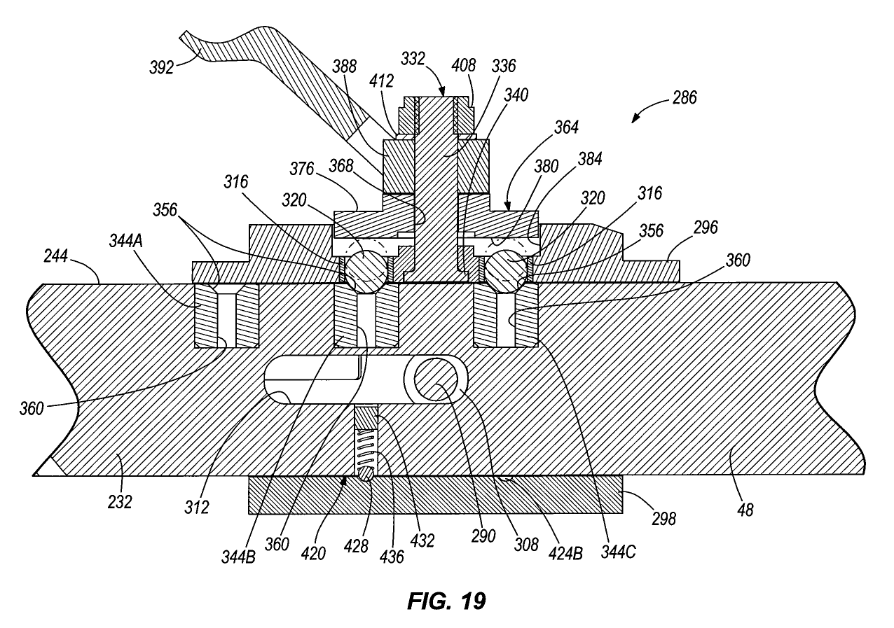

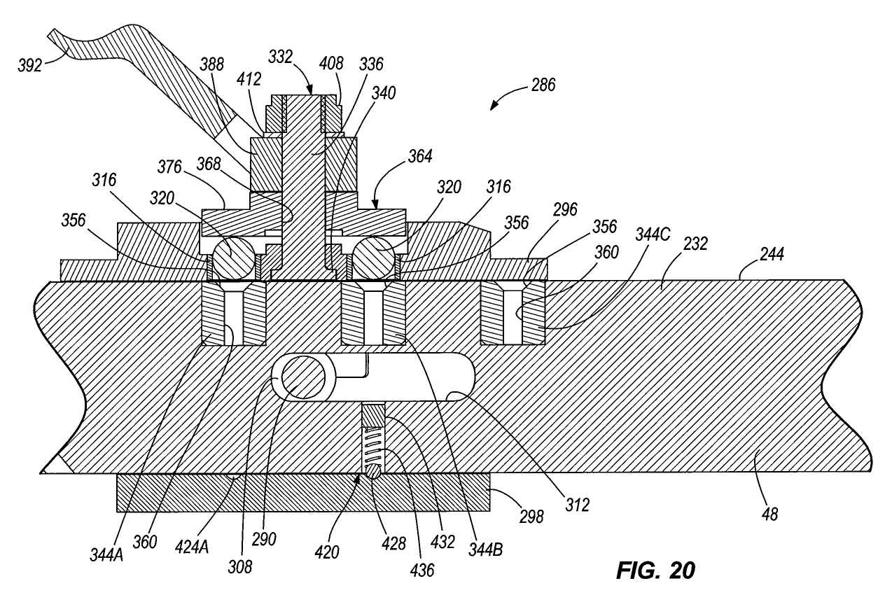

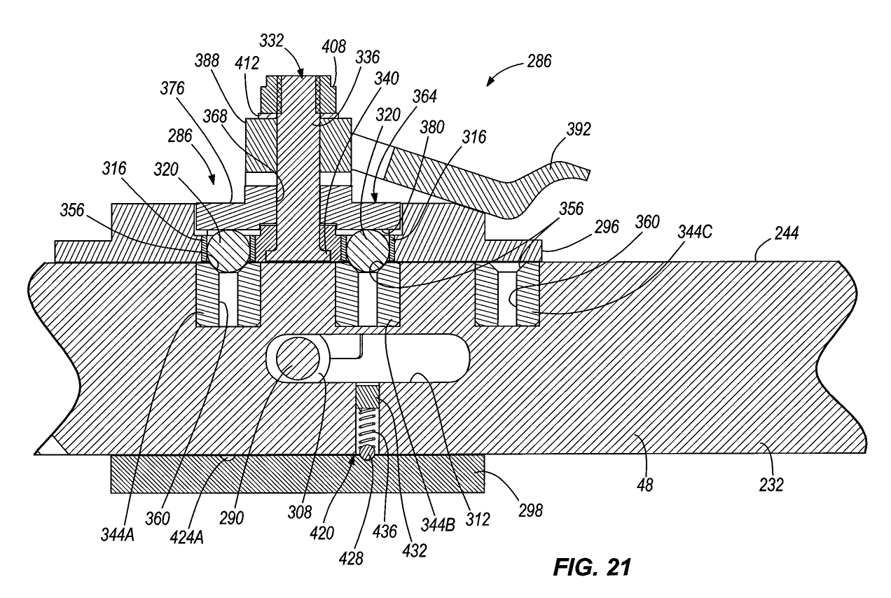

To move the fulcrum adjustor 286 from the fast condition to the second or easy condition, the lever 392 is pivoted upward (as shown in FIG. 19). This upward pivoting of the lever 392 relieves pressure on the plate 364, thereby relieving pressure on the balls 320. With this reduced pressure, a user can move the fulcrum adjustor 286 along the handle 48 to the easy condition illustrated in FIG. 20. Movement of the fulcrum adjustor 286 toward the easy condition causes the balls 320 to ramp up and out of the ball receiving openings 356 of the second and third cups 344B, 344C, roll along the top surface 244 of the handle 48, and move into the ball receiving openings 356 of the first and second cups 344A, 344B. Also with this movement of the fulcrum adjustor 286 to the easy condition, the shaft 290 moves forward in the slot 312 toward the front of the handle 48. The lever 392 is then pivoted downward as shown in FIG. 21 to secure the fulcrum adjustor 286 in the easy condition. Once again, the lever 392 applies a downward force on the plate 364, which applies a downward force on the balls 320. The downward force on the balls 320 forces the balls 320 into the ball receiving openings 356 of the first and second cups 344A, 344B. This downward force is sufficient to retain the fulcrum adjustor 286 in the easy condition. With the balls 320 in the first and second cups 344A, 344B, the fulcrum 288 is in the furthest inward position on the handle 48, thereby providing the user with the longest possible leverage portion of the handle 48 and a minimum stroke length of the piston 44. The longest leverage requires the least amount of force to actuate the handle 48. However, the minimum piston stroke distance emits the least possible amount of mastic per handle stroke. In the easy condition, a user has an easier time pumping the handle 48 (due to the reduced force required to actuate the handle), but more strokes of the handle 48 are required to fill the mastic application tool. Appropriately, this condition is called the “easy” condition. A complete stroke of the handle 48 and piston 44 in the easy condition emits approximately 30 cubic inches of mastic. Again, as an example, reference is made to the mastic application tool disclosed in U.S. Pat. No. 6,874,557. Approximately seven complete strokes of the handle 48 will fill such a mastic application tool when the fulcrum adjustor 286 is in the easy condition.

With continued reference to FIGS. 18-21, the fulcrum adjustor 286 includes a detent mechanism 420 to assist a user in identifying when the fulcrum adjustor 286 is properly positioned in the desired one of the fast and easy conditions. The detent mechanism 420 includes a pair of recesses 424A, 424B defined in an interior surface of the lower housing 298, a spring loaded ball 428, and a spring loaded ball housing 432 supporting a spring 436 and the ball 428. The ball 428 snaps into the appropriate recess 424A or 424B when moved into the desired one of the fast and easy conditions, thereby providing an audible and tactile indication that the fulcrum adjustor 286 is properly positioned and the lever 392 can be moved downward to the locked position.

General operation of the mastic pump 20 will now be described. A user attaches the desired accessory 28 to the pump head 40 by aligning the accessory portion 260 of the accessory connector 52 with the nozzle 258, positioning the connector housing 264 over the nozzle 258, positioning the locking tab 268 in the tab receptacle 274, and actuating the cam levers 266 to the position illustrated in FIG. 12. The user selects either the fast condition or the easy condition as desired. Reference is made to the above description for operation of the fulcrum adjustor 286. The user connects the desired mastic application tool to the accessory 28 and places one foot on the horizontal portion 280 of the foot stand 56. With one hand gripping the mastic application tool and the other hand gripping the handle 48, the user begins to actuate the handle 48. With a starting position as illustrated in FIG. 1, the user moves the handle 48 downward, thereby causing the piston 44 to move upward. When the piston 44 moves upward, the piston diaphragm 208 is in the closed position and the foot valve diaphragm 100 is in the open position. Mastic in the pump body 32 above the piston body 200 is forced out of the pump body 32 through the nozzle 258 and into the mastic application tool via the accessory 28. As the piston 44 moves upward, mastic is drawn from the container 24 through the cavities 124 defined in the valve body 76, through the screen 84 and the three openings 128 in the bottom surface 132 of the valve body 76, past the foot valve diaphragm 100, and into the pump body 32 below the piston valve 184. After the pump handle 48 has been moved downward and the piston 44 moved upward as shown in FIG. 4, the user pulls the handle 48 upward, thereby moving the piston 44 downward. When the piston 44 moves downward, the piston diaphragm 208 is in the open position and the foot valve diaphragm 100 is in the closed position. Mastic positioned in the pump body 32 below the piston valve 184 moves through the piston apertures 216, past the piston diaphragm 208, and into the pump body 32 above the piston valve 184. Mastic is not forced out of the pump body 32 through the foot valve 36 during downward movement of the piston 44 because the foot valve diaphragm 100 is in the closed position. After the handle 48 is moved upward and the piston 44 downward to the position illustrated in FIG. 1, the user repeats the downward movement of the handle 48 as described above. This upward and downward movement of the handle 48 is repeated until the mastic application tool is adequately filled with mastic.

It should be understood that a large variety of alternatives and variations exist to the embodiments discussed above and illustrated in the drawings. One of ordinary skill in the art will be able to recognize such alternatives and variations from the disclosure herein and, therefore, such alternatives and variations are within the spirit and scope of the present invention.

What is claimed is:

1. A mastic pump comprising:
a pump body for holding mastic;
a piston at least partially positioned in the pump body and movable within the pump body;
an actuator coupled to the piston for moving the piston; and
a fulcrum adjustor coupled to the actuator at one of at least two positions.

2. The mastic pump of claim 1, wherein the fulcrum adjustor is movable along the actuator.

3. The mastic pump of claim 1, wherein the fulcrum adjustor includes a lever movable between a locked position, in which the fulcrum adjustor cannot move between the at least two positions, and an unlocked position, in which the fulcrum adjustor is movable between the at least two positions.

4. The mastic pump of claim 3, wherein the fulcrum adjustor includes a ball, a first cup supported by the actuator, and a second cup supported by the actuator, wherein the ball is selectively positionable in one of the first and second cups, movable between the first and second cups when the lever is in the unlocked position, and retained in one of the first and second cups when the lever is in the locked position.

5. The mastic pump of claim 4, wherein the fulcrum adjustor includes a plate, and, when the lever is in the locked position, the lever applies a force to the plate and the plate applies a force to the ball to force the ball into and retain the ball in one of the first and second cups.

6. The mastic pump of claim 5, wherein, when the lever is in the unlocked position, the force applied to the plate by the lever is reduced and the force applied to the ball by the plate is reduced, thereby allowing the ball to move out of the one of the first and second cups.

7. The mastic pump of claim 4, wherein the ball is a first ball, the fulcrum adjustor further comprising a third cup supported by the actuator and a second ball, wherein the first and second balls are selectively positionable in two of the first, second, and third cups, movable between the first, second, and third cups when the lever is in the unlocked position, and retained in two of the first, second, and third cups when the lever is in the locked position.

8. The mastic pump of claim 1, wherein the fulcrum adjustor includes a housing having a first recess and a second recess, the fulcrum adjustor also including a ball and a spring engaging the ball, and wherein the ball is biased by the spring into the first recess when the fulcrum adjustor is in the first position and wherein the ball is biased by the spring into the second recess when the fulcrum adjustor is in the second position.

9. The mastic pump of claim 1, further comprising a fulcrum coupled between the pump body and the fulcrum adjustor.

10. The mastic pump of claim 9, wherein a first end of the fulcrum is pivotally coupled to the pump body and a second end of the fulcrum is pivotally coupled to the fulcrum adjustor.

11. A mastic pump comprising:
a pump body for holding mastic;
a piston at least partially positioned within the pump body and movable within the pump body;
an actuator coupled to the piston for moving the piston; and
a valve diaphragm coupled to the pump body and having an opening defined therein to allow mastic flow.

12. The mastic pump of claim 11, wherein the valve diaphragm is movable between an open position, in which mastic is able to flow by the valve diaphragm, and a closed position, in which mastic is not able to flow by the valve diaphragm, and wherein the opening is blocked to inhibit mastic flow when the valve diaphragm is in the closed position.

13. The mastic pump of claim 11, wherein the opening is defined in an edge of the valve diaphragm.

14. The mastic pump of claim 11, wherein the opening is a first opening, the valve diaphragm further including a second opening.

15. The mastic pump of claim 14, wherein the first and second openings are defined in an edge of the valve diaphragm.

16. The mastic pump of claim 11, further comprising a valve body and a post, the valve body being coupled to the pump body and the post being coupled to the valve body, the valve diaphragm movable along the post between the open and closed positions.

17. The mastic pump of claim 16, wherein the valve body is removably couplable to the pump body without the use of tools.

18. A mastic pump comprising:
a pump body at least partially positionable in a mastic filled container; and
a foot stand coupled to the pump body and including a stepping portion and an upright portion extending vertically upward from the stepping portion, the upright portion including an interior surface that is at least partially concave.

19. The mastic pump of claim 18, wherein a substantial portion of the interior surface of the upright portion is concave.

20. A mastic pump comprising:
a pump body for holding mastic;
a piston at least partially positioned in the pump body and movable within the pump body; and
an actuator coupled to the piston and being movable between a first position and a second position to move the piston, the actuator including a grip that is grippable by a user to move the actuator between the first and second positions, the actuator also including a carrying member spaced from the grip that is grippable by a user to transport the pump.

21. The mastic pump of claim 20, wherein the carrying member is positioned so that the piston does not move upward when the mastic pump is lifted and transported by the carrying member.

22. The mastic pump of claim 20, wherein the carrying member extends from a surface of the actuator.

23. The mastic pump of claim 20, wherein the carrying member extends vertically upward from a top surface of the actuator.

24. The mastic pump of claim 20, wherein a portion of the carrying member that is grippable by a user is non-parallel with a top surface of the actuator.

25. The mastic pump of claim 20, wherein the carrying member includes a first leg and a second leg extending from a top surface of the actuator and being spaced from one another, the carrying member also including an intermediate member coupled to and extending between the first leg and the second leg, the intermediate member being spaced from the top surface of the actuator.

26. The mastic pump of claim 25, wherein the intermediate member is non-parallel to the top surface of the actuator.

27. The mastic pump of claim 20, wherein the grip is positioned near a first end of the actuator and the carrying member is positioned near a second end of the actuator.

28. The mastic pump of claim 20, wherein at least a portion of the carrying member is positioned above the pump body.

29. The mastic pump of claim 20, wherein a longitudinal axis extends through a center of the actuator and a portion of the grip curves away from the longitudinal axis.

30. The mastic pump of claim 20, wherein a longitudinal axis extends through a center of the actuator and the grip includes an arcuate portion that curves away and back toward the longitudinal axis.

31. A mastic pump comprising:
a pump body for holding mastic;
a piston at least partially positioned in the pump body and movable within the pump body;
an actuator coupled to the piston for moving the piston; and
an accessory connector coupled to the pump body for connecting and disconnecting a mastic tool accessory to the pump body without the use of tools.

32. The mastic pump of claim 31, wherein the accessory connector includes a lever movable between a locked position and an unlocked position.

33. The mastic pump of claim 31, wherein the accessory connector includes two levers, each lever movable between a locked position and an unlocked position.

34. The mastic pump of claim 31, wherein the accessory connector includes a nozzle connected to the pump body and an accessory portion connected to a mastic tool accessory, the nozzle and the accessory portion being connectable to and disconnectable from each other without the use of tools to respectively connect and disconnect a mastic tool accessory to the pump body.

35. The mastic pump of claim 34, wherein one of the nozzle and the accessory portion includes a lever that is movable between a locked position, in which the mastic tool accessory is connected to the pump body, and an unlocked position, in which the mastic tool accessory is disconnected from the pump body.

36. The mastic pump of claim 35, wherein the accessory portion includes the lever.

37. The mastic pump of claim 34, wherein one of the nozzle and the accessory portion includes a pair of levers that are movable between a locked position, in which the mastic tool accessory is connected to the pump body, and an unlocked position, in which the mastic tool accessory is disconnected from the pump body.

38. The mastic pump of claim 34, wherein the nozzle includes a groove and the accessory portion includes a lever capable of pivoting between a locked position and an unlocked position, wherein a portion of the lever is positioned in the groove when the lever is in the locked condition to connect the mastic tool accessory to the pump body, and wherein the portion of the lever is not positioned in the groove when the lever is in the unlocked condition to disconnect the mastic tool accessory from the pump body.

39. The mastic pump of claim 31, wherein one of the accessory connector and the pump body includes a projection and the other of the accessory connector and the pump body includes a receptacle, the projection being positioned in the receptacle when the accessory connector is coupled to the pump body.

40. The mastic pump of claim 39, wherein the accessory connector includes the projection and the pump body includes the receptacle.

41. The mastic pump of claim 39, wherein rotation of the accessory connector is inhibited when the projection is positioned in the receptacle.
